(19) United States
(12) Patent Application Publication
(10) Pub. No.: US 2001/0015208 A1
(43) Pub. Date: Aug. 23, 2001

(54) SNORT RESTRAINING DEVICE

(76) Inventor: OSAMU KONISHI, HACHIOJI-SHI (JP)

Correspondence Address: BIRCH STEWART KOLASCH & BIRCH, PO BOX 747, FALLS CHURCH, VA, 22040-0747, US

(21) Appl. No.: 09/083,086

(22) Filed: May 22, 1998

(30) Foreign Application Priority Data
Oct. 13, 1997 (JP) 9-294971
Dec. 30, 1997 (JP) 9-368240

(51) Int. Cl. A61F 5/56

(52) U.S. Cl. 128/848

(57) ABSTRACT

A snort restraining device comprising a gas bag receiving mainly therein air, nitrogen or carbon dioxide and having an elongated configuration, and a mounting device for mounting the gas bag generally along the longitudinally central portion of the back of a person during the sleeping condition of the person, whereby the upward facing sleeping attitude is prevented reliably thereby the snort is restrained.

### BACKGROUND OF THE INVENTION

1. Field of the Invention

This invention relates to snort restraining device and, more particularly to an improved device effective in preventing or reducing the snort.

2. Description of Prior Art

The snort is defined as rattling or grunting noise often made by sleepers in breathing, and particularly, as breathing noise when the breathing air passes through the upper airway or the air passage between the nose and the pharynx. It has been verified that the snort is liable to cause when the upper airway is strictured, and the stricture of the upper airway is caused mainly by downward movement of the base portion of the tongue or of the soft palate when the person is sleeping with one's face upward. Accordingly, there is a deep connection between the snort and the sleeping attitude, and it is possible to restrain the snort by taking side facing sleeping attitude.

There have been proposed various snort restraining devices for maintaining side facing sleeping attitude. Among them, a device named as ┌Snort Restraining Pillow┘ has been made for practical use and has got popularity. The pillow have different heights between the left and the right ends and, when the user wishes to sleep with his right arm being maintained upperside, the pillow is located with the right end thereof being higher than the left end. Conversely, when the user wishes to sleep with his left side maintained upper, the pillow is located with the left end being higher than the right end. The pillow is effective in keeping the side-facing sleeping attitude, by preventing the turn over during the sleep of the user, and thereby preventing the snort.

However, a person usually changes his sleeping attitude ten times or more during the sleep of one night, thus, even though the side facing sleeping attitude is kept initially by the ┌Snort Restraining Pillow┘, the person tends to remove the pillow and, to sleep with his face upward. Thus, the pillow is effective to prevent the snort for only a relatively short time period.

### BRIEF SUMMARY OF THE INVENTION

1. Purpose of the Invention

Accordingly, an object of the invention is to provide a device effectively restraining the snort by forcefully maintaining the side facing sleeping attitude throughout the sleep.

Another object of the invention is to provide a device effectively restraining the snort by positively preventing the upward facing sleeping attitude.

2. Brief Description of the Invention

According to the invention, there is provided a device comprising an air bag and means for locating the air bag to the back of a person during the sleeping condition of the person.

The locating means may be night clothes or pajamas having on the back portion thereof a pocket for receiving therein the air bag.

The locating means may be a combination of a pocket, a belly band and shoulder bands with the pocket being connected to the belly band and the shoulder bands for maintaining the pocket at a desired position, and the pocket being adapted to receive the air bag.

Preferably, the belly band has a substantial width, but the belly band may be substituted by a waist belt with the shoulder bands being connected to the upper end of the pocket and the lower end of the pocket being connected to the waist belt.

Preferably, the air bag is provided with an air plug for adjusting the amount and the pressure of the air received therein.

The air bag may be formed of elastomer material such as a natural rubber, synthetic rubber and the like.

When the air bag is formed of elastomer material, it is preferable to provide suitable reinforcing member such as textile and the like.

The air bag may be formed of any suitable air tight material such as synthetic resin material, rubberized cloth and the like.

A gas filled bag may be used instead of the air bag, and the gas filled therein may be nitrogen, carbon dioxide and the like.

### BRIEF DESCRIPTION OF THE DRAWING

In the accompanying drawings, there is shown some of various possible embodiments of the present invention:

FIG. 1 is a perspective view as viewed from the frontside of a snort restraining clothes such as pajamas coat having a pocket for receiving therein an air bag according to a first embodiment of the invention;

FIG. 2 is a detailed perspective view showing the pocket of FIG. 1 being detachably mounted on pajamas coat of FIG. 1.

FIG. 3 is a perspective view as viewed from the backside of a snort restraining undergarment according to a second embodiment of the invention;

FIG. 4 is a perspective view as viewed from the backside of a snort restraining undergarment according to a third embodiment of the invention, and

FIG. 5 is a perspective view as viewed from the frontside of a snort restraining undergarment according to a fourth embodiment of the invention.

### DETAILED DESCRIPTION OF PREFERRED EMBODIMENTS

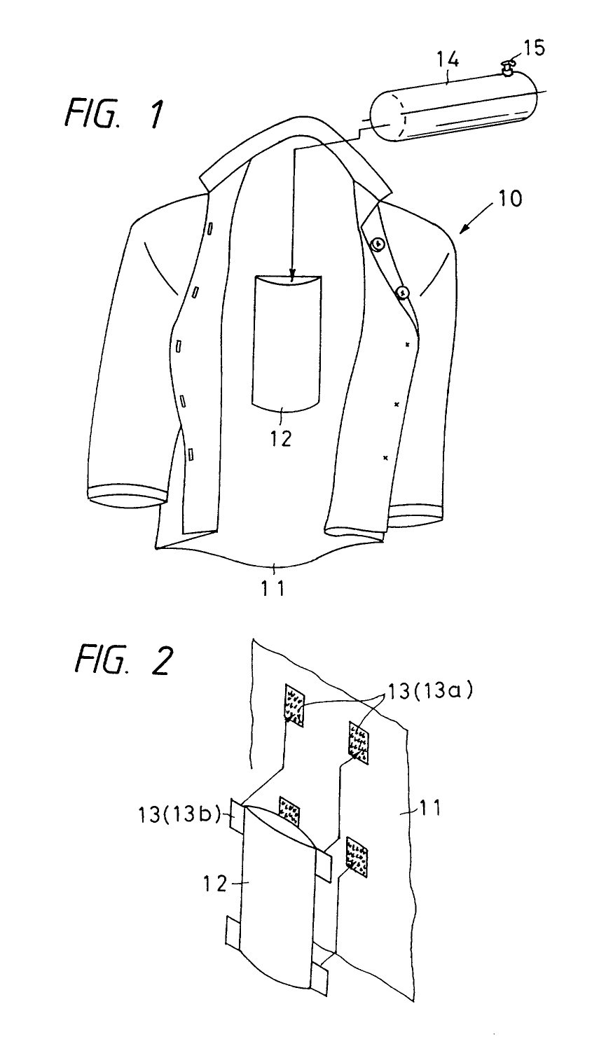

FIG. 1 shows a snort restraining undergarment such as an upper garment 10 of pajamas according to a first embodiment of the invention, in which, a pocket 12 is provided on the central portion of the inside of the back body 11 of the garment 10. In FIG. 2, the pocket 12 is detachably connected to the garment 10 and, fasteners 13 constituted of male fasteners 13a and female fasteners 13b are utilized. The male fasteners 13a are provided on the garment 10 and the female fasteners 13b are connected to the pocket 12. The fasteners 13 may be substituted by other publicly known connecting means such as snap buttons, buttons and button holes and the like. Further, the pocket 12 may permanently be connected to the garment 10. The pocket 12 may be provided on the outside of the back body 11.

The pocket 12 is adapted to receive snugly therein an air bag 14 according to the invention The air bag 14 is formed of any suitable air tight flexible material such as rubber, synthetic resin material and the like, and an air plug 15 is provided for adjusting the amount and the pressure of the compressed air received in the air bag 14. The air bag 14 may be substituted by a gas filled bag receiving therein such as nitrogen or carbon dioxide. The air bag 14 may have any desired cross-section such as a circle, an eclipse and the like.

In use, the person wears the garment 10 mounting thereon the air bag 14. The air bag 14 takes a position extending generally along the spinal column of the wearer, and effectively prevents the upward facing sleeping attitude and, the person is forced to take a side facing sleeping attitude. Thus, the downward movement of the base portion of the tongue or of the soft palate snort is prevented effectively and the stricture of the upper airway is also prevented, thereby the snort is prevented effectively.

Further, when the wearer's body makes a relatively small turn over, the airbag 14 acts to disturb the turn of the body, and the wearer tends to take the same side facing sleeping attitude as before.

And, since the wearer's muscle cannot endure to keep the same sleeping attitude for a long time, the wearer's body begin to make a big and strong turnover and want to take the upward facing sleeping attitude, in spite of the disturbance of the air bag 14, during the deep sleep unconsciously, and the wearer's body takes a position to mount on the air bag 14. But, this status is very unstable, and if the body turns a little in either rightward or leftward direction, the air in the air bag 14 begins to move, and the shape of the air bag changes to help the turn, and the wearer's body is forced to roll over the air bag smoothly, and to take a side facing sleeping attitude.

In every turn over of the body, the same procedure is repeated, and the side facing sleeping attitude is maintained throughout the whole sleeping time period. Thus, the snort is restrained effectively.

Further, the air bag 14 can act as a cushion, which helps a comfortable sleep when the wearer takes a side facing sleeping attitude.

Further, as the stricture of upper airway is restrained during the side facing sleeping attitude, this device is effective in improving the insufficient respiratory syndrome.

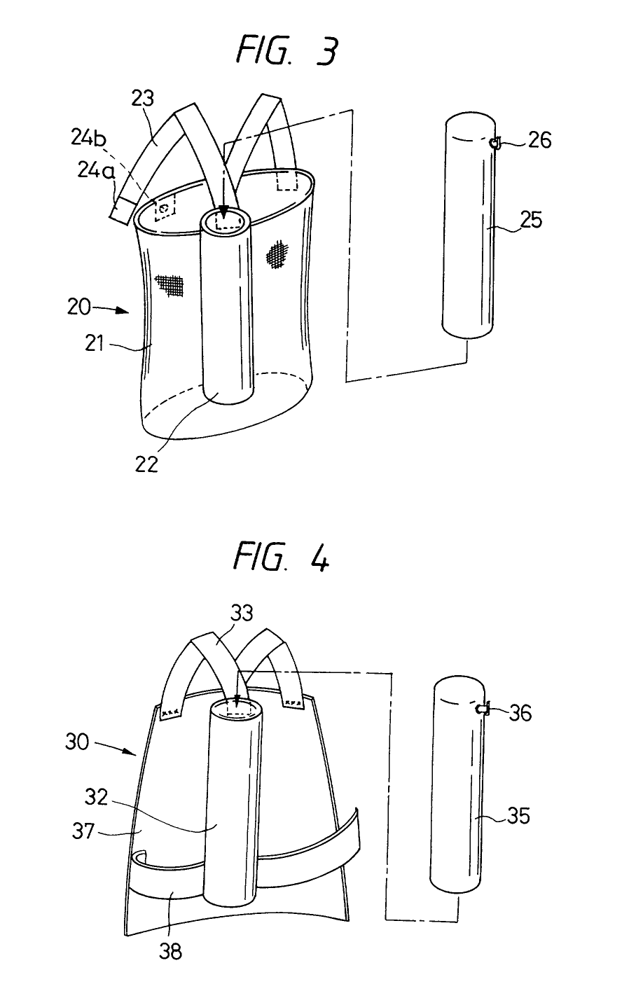

FIG. 3 shows the second embodiment of the present invention, in which, a pair of shoulder straps 23 and a generally vertically extending tubular pocket 22 are connected to a belly belt 20 having a substantial width One end of each strap 23 is detachably connected to the belly belt 20 through detachable connecting devices 24a and 24b such as snap button and the like. The straps 23 may be an extendable type.

Similar to the first embodiment, an air bag 25 is snugly received in the pocket 22. An air plug 26 is provided on the air bag 25 for adjusting the elasticity of the air bag 25. The air bag 25 having the air plug 26 may be substituted by a gas filled bag.

In use, the shoulder straps 23 act to maintain the air bag 25 at the position generally extending along the spine of the sleeping person, thus, upward facing sleeping attitude can reliably be prevented, and the snort can reliably be avoided.

FIG. 4 shows the third embodiment of the present invention, in which, a pair of shoulder straps 33 and a waist belt 38 are connected to an abdomen cover 37, a generally vertically extending tubular pocket 32 is connected to a belly belt 38, and an air bag 35 is received in the tubular pocket 32. Preferably, the shoulder straps of each strap 33 and the belly belt 38 are detachably connected to the abdomen cover 37 through detachable connecting devices such as snap buttons and the like. The pocket 32 is adapted to receive snugly an air bag 35 which is similar to the air bags 15 and 25. An air plug 36 is provided on the air bag 35.

When a person wears the device of FIG. 4, the abdomen cover generally covers the abdomen of the person and the pocket 32 generally extends along the spine, thus, the upward facing sleeping attitude can effectively be prevented and, the snort can reliably be restrained.

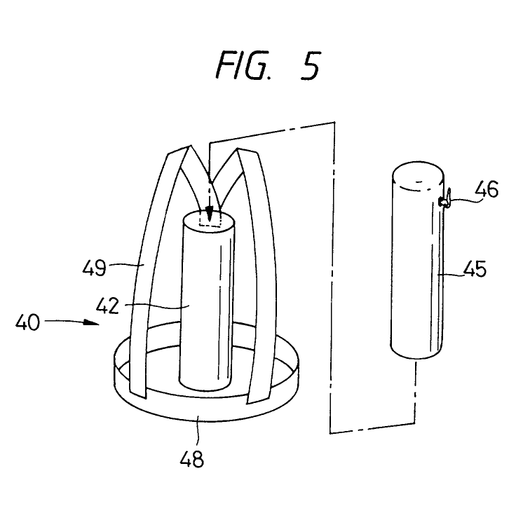

FIG. 5 shows the fourth embodiment of the present invention, in which, a pair of shoulder straps 49 and a waist belt 48 are connected to a generally vertically extending tubular pocket 42. Although it is not shown clearly in the drawing, the waist belt 48 is connected to the lower end portion of the tubular pocket 42. An air bag 45 is adapted to be received in the tubular pocket 42. The shoulder straps 49 and the waist belt 48 may detachably be connected to the tubular pocket 45. The pocket 42 is adapted to receive snugly an air bag 45 which is similar to the air bags 15 and 25. An air plug 46 is provided on the air bag 45 for adjusting the pressure of the compressed air received in the air bag 45.

When a person wears the device of FIG. 5, the pocket 42 receiving therein the air bag 45 extends generally along the spine, thus, the upward facing sleeping attitude can effectively be prevented, and the snort can reliably be restrained.

As described above, the device according to the invention comprises an air bag or a gas filled bag having an elongated shape and means for mounting the bag along the longitudinally central portion of the back of a person, whereby the upward facing sleeping attitude is prevented reliably and the snort is prevented.

As described heretofore in detail, the snort restraining device according to the invention comprises a gas bag having an elongated shape and means for locating the gas bag generally along the longitudinally central portion of the back of a person during the sleeping condition of the person, whereby the upward facing sleeping attitude is prevented reliably thereby the snort is restrained.

Typically, the gas bag is filled mainly with a gas selected from the group of air, nitrogen and carbon dioxide, or the mixture of these gasses with any other gases.

Preferably, the gas bag is an air bag, and an air plug is provided for adjusting the amount and the pressure of the air received in the air bag, and the air may include exhaled air, with which the user can simply expand the air bag by blowing air through his mouth.

Preferably, the gas bag is formed of elastomer material such as a natural rubber, synthetic rubber and the like.

Further, the gas bag may be provided with reinforcing member such as textile, metal wires and the like.

Means for mounting the gas bag may be an upper garment of pajamas having a pocket on the central portion of the back body of pajamas, with the pocket being adapted to receive the gas bag.

The upper garment of pajamas may be substituted by a combination of a cylindrical pocket, and a belly belt and a pair of shoulder straps being connected to the cylindrical pocket and adapted to maintain the pocket generally along the longitudinally central portion of the back of a person, with the pocket being adapted to receive the gas bag.

The upper garment of pajamas may be substituted by a cylindrical pocket, and an abdomen cover, a belly belt and a pair of shoulder straps being connected to the cylindrical pocket and adapted to maintain the pocket generally along the longitudinally central portion of the back of a person, with the pocket being adapted to receive the gas bag.

Further, the upper garment of pajamas may be substituted by a cylindrical pocket, and a waist belt and a pair of shoulder straps being connected to the cylindrical pocket and adapted to maintain the pocket generally along the longitudinally central portion of the back of a person, with the pocket being adapted to receive the gas bag.

The pocket may have any desired cross-section such as circular, oval and the like.

The invention is by no means limited to the form of embodiments described and illustrated which have been given by way of example only. In particular, it comprises all means constituting technical equivalents to the means described as well as their combinations, should the latter be carried out according to its gist and within the scope of the following claims.

What is claimed is:

1. A snort restraining device comprising a gas bag having an elongated shape and means for locating the gas bag generally along the longitudinally central portion of the back of a person during the sleeping condition of the person, whereby the upward facing sleeping attitude is prevented reliably thereby the snort is restrained.

2. A snort restraining device according to claim 1, in which the gas bag is an air bag, and an air plug is provided for adjusting the amount and the pressure of the air received in the air bag.

3. A snort restraining device according to claim 1, in which the gas bag is filled mainly with a gas selected from the group of air, nitrogen and carbon dioxide.

4. A snort restraining device according to claim 1, in which the gas bag is formed of elastomer material such as a natural rubber, synthetic rubber and the like.

5. A snort restraining device according to claim 1, in which the gas bag is provided with reinforcing member such as textile, metal wires and the like.

6. A snort restraining device according to claim 1, in which means for mounting the gas bag is an upper garment of pajamas having a pocket on the central portion of the back body of pajamas, said pocket being adapted to receive said gas bag.

7. A snort restraining device according to claim 1, in which said means for mounting the gas bag comprises a cylindrical pocket, and a belly belt and a pair of shoulder straps being connected to the cylindrical pocket and adapted to maintain the pocket generally along the longitudinally central portion of the back of a person, said pocket being adapted to receive the gas bag.

8. A snort restraining device according to claim 1, in which said means for mounting said gas bag comprises a cylindrical pocket, and an abdomen cover, a belly belt and a pair of shoulder straps being connected to the cylindrical pocket and adapted to maintain the pocket generally along the longitudinally central portion of the back of a person, said pocket being adapted to receive said gas bag.

9. A snort restraining device according to claim 1, in which said means for mounting the gas bag comprises a cylindrical pocket, and a waist belt and a pair of shoulder straps being connected to the cylindrical pocket and adapted to maintain the pocket generally along the longitudinally central portion of the back of a person, said pocket being adapted to receive said gas bag.

10. A snort restraining device according to either one of claims 6 through 9, in which said pocket has a generally circular cross-section.

11. A snort restraining device according to either one of claims 6 through 9, in which said pocket has a generally oval cross-section.
